(19) United States
(12) Patent Application Publication
(10) Pub. No.: US 2008/0233208 A1
(43) Pub. Date: Sep. 25, 2008

(54) USE OF ERIANIN IN PREPARING PHARMACEUTICAL FOR TREATING TUMORS

(75) Inventors: Yiping Li, Taizhou City (CN); Ning Li, Taizhou City (CN); Danping Zhou, Taizhou City (CN); Lizuan Chen, Taizhou City (CN)

Correspondence Address: OCCHIUTI ROHLICEK & TSAO, LLP, 10 FAWCETT STREET, CAMBRIDGE, MA, 02138, US

(73) Assignee: The Jiang Cell Biomedical Research Co., Ltd., Taizhou City (CN)

(21) Appl. No.: 12/023,813

(22) Filed: Jan. 31, 2008

(30) Foreign Application Priority Data
Feb. 1, 2007 (CN) 200710063473.6

(51) Int. Cl. A61K 31/09; A61P 35/00; A61K 33/24; A61K 31/505; A61K 31/704; A61K 31/195; A61K 31/337; A61K 31/4545

(52) U.S. Cl. 424/649; 514/721; 514/274; 514/34; 514/564; 514/449; 514/283

(57) ABSTRACT

This invention relates to a use of the compound of formula (I), Erianin, in preparing pharmaceutical for treating tumors

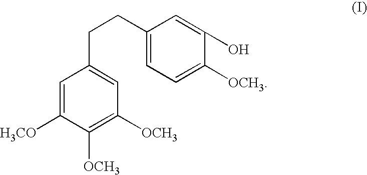

### FIELD OF INVENTION

This invention relates to a new use of Erianin.

This invention also relates to a method for treating tumors.

### BACKGROUND OF INVENTION

Cancer is a disease that seriously jeopardizes the health of human beings. Around the globe, about 6 million people die of cancer every year, with another 10 million on the brink of death. According to the estimate of the World Health Organization, in the 21^{st} century, cancer will become the “number one killer” of mankind. The national retrospective investigation on the causes of death showed that in the past two decades, the morbidity and death rate of cancer in China have been on the rise year by year. Out of every five people died of disease, one was of cancer; for every 200 families, one suffered the pain of having a family member with cancer.

In the past several decades, many ways of treating cancer became available, mainly including surgery, radiotherapy, chemotherapy, hormonotherapy, gene therapy, and immunotherapy, among which surgery, radiotherapy and chemotherapy have became the major means. Chemotherapy refers to treating cancer with chemical medication. It is the field of the most rapid development in the diagnosis and treatment of cancer. A great number of new medicines aiming at different targets are ready for clinical application, and developments in research in mechanism of drug action and pharmacokinetics have made the clinical administration routes and means more fitting for killing tumor cells while protecting the normal tissues.

At present, pharmaceuticals for chemotherapy mainly includes: pharmaceuticals that affects the biosynthesis of nucleic acid, e.g. fluorouracil, 6-oxypurine, amethopterin, cytarabine, hydroxyurea; pharmaceuticals that directly destroys DNA and prevents its reproduction, e.g. alkylating agents; antineoplastic antibiotics, e.g. Cisplatin and Carboplatin; pharmaceuticals that interferes with the transcription and prevents the synthesis of RNA, e.g. actinomycin D, adriamycin, and other transcription restraining antibiotics; pharmaceuticals that affects the synthesis of protein, e.g. catharanthines, podophyllotoxins, harringtonine, asparaginase; hormones, e.g. adrenal cortical hormone, estrogen, androgen, tamoxifen, aminoglutethimide. The existing chemotherapies and radiotherapies that are commonly used in treating cancer may cause serious toxic and other side effects that are adverse to the human body.

The property of interfering the polymerization or depolymerizaion of microtubulin of many natural medicines is regarded as having antineoplastic activity. Such medicines include vincristine, taxanes, and macrolide antineoplastic drugs. Microtubules play an important role in cell division, and the development of microtubulin binding factors is based on their capability of interfering cell proliferation. The depolymerizing factor of microtubulin, such as colchicine and vincristine, has the antimitotic effect, causing the tumor vessels to close. However, such effect of closing the tumor vessels only occurs when the dosage approaches the MTD (maximum tolerated dose). Endostatin, the latest discovered inhibiting factor of vasculogenesis, has the inhibiting effect on new vasculogenesis, but has no obvious effect on existing vessels or obvious target effect on tumor vessels.

A new type of microtubulin depolymerizing factor discovered in recent years can solve this problem by closing the vessels with dosage well below the MTD (Expert Opin Investig Drugs. September 2004; 13 (9) 1171-82). In 2005, Loïc Vincent et al mentioned a new type of microtubulin depolymerizing factor with similar property destroying microtubulin skeleton as vascular targeting factors (VTAs). Literature data shows that VTAs can selectively induce the decay of tumor vessels, partly through the VE-cadherin signal channel. This kind of microtubulin depolymerizing factor selectively destroys tumor vessels and prevent new vasculogenesis in the tumor while having no influence on the normal vascular system. At the same time, it can inhibit the polymerization of microtubulin, selectively cause tumor vessel function disorder and structural damage, and induce the apoptosis of vessel endothelial cells, playing its role of killing tumor cells or inhibiting tumor metastasis by making tumor cells lose the support of nutrition and oxygen.

In 2005, Gillian M. Tozer et al reported in an influential journal, Nature Rev Cancer, that this kind of compound not only affects the proliferation of endothelial cells, but also influences their transposition, quickly changing the pattern of endothelial cells, causing their apoptosis, and breaking their connections, so as to immediately cause the tumor vessel function disorder and structural damage. As the normal vessels are generally supported by smooth muscle cells, this kind of compound only affects the vessels without such support and has no influence on vessels supported by smooth muscle cells, it can quickly and selectively cause the tumor vessel function disorder and structural damage, thus affecting the tumor cells selectively (Nat Rev Cancer. June 2005; 5(6) 423-35, J. Clin. Invest., Nov. 1, 2005; 115(11): 2992-3006). At present, this type of medication is regarded as one of the most promising antineoplastic medicines.

Now Combretastatin A-4 is the only medicine of this kind that is under research overseas, whose research has entered the clinical stage. Through double key connection, the most effective configuration in the stilbene units of Combretastatin to destroy tumor vessels is cis-configuration, and the stilbene compound of tran-sconfiguration has no inhibiting effect on tumors. It is very easy for reactions such as isomerization to take place due to the existence of cis trans isomerism. The tran-sconfiguration not only lacks efficacy, but also brings certain toxic side effects at the same time (for Combretastatin A-4, LD50 is 500 mg/kg), resulting in great difficulty in the preservation and actual application of Combretastatin A-4.

In this invention, Erianin is a synthesized compound, its structural formula being formula (I):

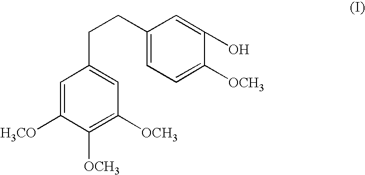

At present, there is related literature reporting the curative effect of Erianin on liver cancer. The findings of Wang Tianshan (In vitro Inhibition Activities on the Growth of Tumor Cell Strain K₂₅₆ by Constituents from Dendrobium Chrysotoxum, Natural Product Research and Development, 1997, 9 (2), 1˜3) showed that bibenzyls and phenanthrenes have inhibitory effect on the assembling and caryocinesis of the in vitro cultured murine microtubulin L1210, P388 cell strain and many human tumor cell strains including A-549, MCF-7, HT-29, SKMEL-5, MLM, SK-OV-3, and HL-60. The dihydrobibenzyls and phenanthrenes in Dendrobium chrysotoxum have different degrees of inhibitory effect on the growth of tumor cell strain K₂₅₆, among which Erianin (dihydrocombretastatin A-4) is the most active.

Erianin has the best effect on the liver cancer of mouse, its tumor inhibitory rate being 50.82%. Related study (Journal of China Pharmaceutical University, 1994, 25 (3), 188˜189) infers that the toxic side effect of Erianin is far lower than 5FU, a medicine for chemotherapeutics of cancer. Erianin has negative proton effect to many kinds of cancer cells, the effecting target being the microtubulin in the cell. It can inhibit the polymerization of microtubulin, stimulate the hydrolysis of microtubulin-dependent GTP, and competitively bind with protein with colchicine. The study of Li Yunman (Erianin Induces Apoptosis of Human leukemia HL-60 cells, Acta Pharmacologica Sinica, November 2001, 22(11),1018-1022) showed that Erianin significantly inhibited the growth of human leukemia HL-60 cells. The inhibition might be the result of the induced apoptosis and the altered expression of bcl-2 and bax genes in HL-60 cells.

This invention relates to the obvious curative effect of Erianin on new cancers through experiments. The Erianin of this invention is obtained through chemical synthesis. As a tumor vessel inhibitor, it is unlike Combretastatin A-4 in that the two benzene rings are connected by a single bond, which results in the differences in structure, conformation, bonding force, and trans effect between Erianin and Combretastatin A-4, greatly increasing the stability of medication (Light may isomerize Combretastatin A-4 to tran-sconfiguration, so it should be kept out of light) and the effect over microtubulin while exerting much lower toxic side effects.

### SUMMARY OF INVENTION

One object of this invention is to provide a use of the compound of formula (I), Erianin, in preparing pharmaceutical for treating tumors.

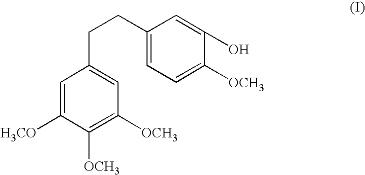

Another object of this invention is to provide a method of treatment of tumors by administering to a subject in need of such treatment a compound of formula (I), Erianin, or a compound of formula (I) in combination with other antitumor agents.

Tumors referred to in this invention may be solid tumors such as sarcomas and cancers (for instance, fibrosarcoma, myxosarcoma, liposarcoma, chondrosarcoma, osteogenic sarcoma, notochordoma, hemangiosarcoma, endotheliosarcoma, lymphangiosarcoma, lymphangioendotheliosarcoma, synovial bursa, mesothelioma, Ewing's tumor, leiomyosarcoma, rhabdomyosarcoma, carcinoma of colon, carcinoma of pancreas, breast cancer, oophoroma, prostatic carcinoma, squamous carcinoma, basaloma, adenocarcinoma, syringocarcinoma, sebaceous carcinoma, papillary carcinoma, papillary adenocarcinoma, cystadenocarcinoma, encephaloid carcinoma, bronchiolar carcinoma, renal cell carcinoma, liver neoplasm, cholangiocarcinoma, choriocarcinoma, seminoma, embryonal carcinoma, Wilms' tumor, cervical carcinoma, uterine cancer, carcinoma of testis, lung cancer, small cell carcinoma of lung, carcinoma of urinary bladder, epithelial cancer, glioma, astrocytic glioma, acoustic tumor, oligodendroglioma, neurinoma, meningeoma, melanoma, neuroblastoma and retinoblastoma), or hematic tumors such as leukemia (for instance, acute leukocythemia, acute lymphoblastic leukemia, acute myeloblastic leukemia, acute myelogenous leukemia, acute promyelocytic leukemia, acute myelomonocytic leukemia, acute monocytic leukemia, acute erythroleukemia, chronic leukemia, chronic myeloblastic leukemia, chronic lymphoblastic leukemia), polycythemia vera, lymphoma (Hodgkin's disease, non-Hodgkin's disease), Waldenstrom's macroglobulinemia, heavy chain disease. And sarcoma, liver cancer, lung cancer, melanoma, leukemia, esophageal cancer, and sdenocarcinoma of stomach are selectively preferable.

Researches show that by inhibiting the polymerization of microtubulin, selectively causing tumor vessel function disorder and structural damage, and inducing the apoptosis of vessel endothelial cells, Erianin plays its role of killing tumor cells by making tumor cells lose the support of nutrition and oxygen.

Erianin may also be jointly used with other therapies such as chemotherapy, surgery, radiotherapy, immunotherapy, anti-vasculogenesis therapy or gene therapy to remarkably enhance the curative effect on tumors. Preferably, the said other therapy is the chemotherapy using antineoplastic drugs. More preferably, the said other therapy is the one using cytotoxin and anti-proliferation and vasculogenesis inhibiting agent, the preferred medicines including Cisplatin, fluorouracil, adriamycin, leuleran, melphalan, taxol, irinotecan CPT-11, Avastin, ZD6126 (N-acetylcochinol-O-phosphate, vascular endothelial-cadherin antibodies anti-E-cadherin (BV13) or against VE-cadherin (E4G10), etc. The property of low toxicity of Erianin enables it to enhance the efficacy of chemotherapeutics without adding to its toxicity, thus showing great prospect in joint application with chemotherapeutics. In an embodiment of this invention in which Erianin was jointly used with Cisplatin, the mice in the testing group of sole Erianin administration and the group of joint administration of Erianin and Cisplatin had normal appetite, bright hair and free movements. No other abnormality was detected and no death occurred. However, the mice in the testing group of cyclophosphamide administration showed the signs of athrepsia, listlessness, and pilo-erection. This phenomenon indicated that the toxicity of Erianin or the joint administration of Erianin and DDP is lower than that of the sole administration of cyclophosphamide.

By using Erianin as the effective ingredient, adding pharmaceutically acceptable accessories, and using the common method of this field, pharmaceutical preparation of this invention can be prepared. The dosage forms of the pharmaceutical can be: oral administration form, such as tablet, capsule (including hard capsule, soft capsule, enteric-coated capsule and microcapsule), powder, granules and syrups; non-oral administration form, such as injection, lyophilization, suppositories, pill, gelata and patch. Besides these common forms, the orally taken speed-release preparation (such as tablet, granules) and the orally or non-orally taken sustained-release preparation (such as tablet, granules, refined granules, pill, capsule, syrup, stable suspension, solution) can also be used in current invention, and preparations thereof are available by common methods. The preparation in current invention can be coated or non-coated according to the need. The injection form and the oral administration form of preparation of Erianin are preferred in this invention.

The medicated accessories in this invention include those for solid preparation, such as excipient, lubricant, cohesives, disintegrant, stabilizer, blowing agent, coating agent, etc; or those for semisolid preparation and liquid preparation, such as solvent, solubilizing agent, suspending agent, isotonizing agent, buffering agent, emollient, emulsifier, etc. In addition, other pharmaceutical additives, such as preservative, antioxidant, colorant, edulcorant, correctant, etc. can also be used if necessary.

The following embodiments are presented to describe the invention without bringing any limitation to the scope of protection of this invention.

### DETAILED DESCRIPTION OF THE INVENTION

### Preparation of Erianin

### Example One

### Preparation of Erianin

The Erianin used in this invention is provided by Zhejiang Cell Biomedical Research CO., LTD., and other test materials are all purchased commercially, unless indicated otherwise.

The detailed process for the preparation of Erianin is as follows:

### 1. The Preparation of 3,4,5-trimethoxybenzylalcohol

3,4,5-trimethoxybenzaldehyde (15 g, 76.45 mmol) and anhydrous alcohol (200 ml) were placed in a 250 ml three-necked flask, and were dissolved at 40° C. Sodium borohydride (1.48 g,38.23 mmol) was added to the solution. The resulting mixture was heated to reflux for 45 minutes, and monitored by TLC. When the reaction is completed, cooling it to room temperature, deionized water (10 ml, 555.8 mol) was added to quench the reaction. After suction filtering, the filter residue was washed by anhydrous alcohol (20 ml), the combined filtrate was concentrated in rotatory evaporater to dry, dichlormethane (100 ml) was added to dissolve the crude product. The organic layer was washed with sodium hydroxide solution (50 ml) twice and with deionized water (50 ml) twice, and a proper amount of anhydrous magnesium sulfate was added to dry it overnight. After filtering, washing the filter residue with dichlormethane (20 ml). The combined filtrate was concentrated in a rotatory evaporater to afford 3,4,5-trimethoxyl benzyl alcohol, 14.05 g of colorless oily product, yield: 92.72%.

The product does not need to be further purified for following reaction. If pure product is wanted, it can be vacuum distilled for the fraction of distillate of BP 216-218° C./12 mmHg.

### 2. The Preparation of 3,4,5-trimethoxyl benzyl bromide

Dissolving 3,4,5-trimethoxyl benzyl alcohol (14.05 g, 70.89 mmol) in dichlormethane (100 ml) in a 250 ml three-necked flask; phosphorus tribromide (6.73 ml, 70.89 mmol) in dichlormethane (25 ml) was added dropwise and allowed to react at room temperature for 50 minutes, cooling in ice bath, slowly adding deionized water (18 ml,1.0 mol) dropwise to quench the reaction, washing with deionized water (100 ml) twice, drying with anhydrous magnesium sulfate, filtering, washing the filter residue with dichlormethane (20 ml), the combined organic layer was concentrated in rotatory evaporater to dry, and was further dried under vacuum to afford 3,4,5-trimethoxyl benzyl bromide (16.05 g of faint yellow solid), yield 84.44%.

The product does not need to be further purified for following reaction. If pure product is wanted, it can be recrystallized to get the white lamellar crystal with a 1:3 mixture of ethyl acetate and n-hexane.

### 3. The Preparation of 3,4,5-trimethoxyl benzyl triphenylphosphine bromide

Dissolving 3,4,5-trimethoxyl benzyl bromide (16.05 g, 61.47 mmol) in toluene (150 ml) in a 250 ml three-necked flask, adding triphenylphosphine (16.12 g, 61.47 mmol) and dissolving immediately. The reaction mixture was heated to reflux for 1 hour, white solid was separated, then cooling to room temperature, suction filtering, the filter cake was washed with toluene (30 ml). After vacuum drying, 3,4,5-trimethoxyl benzyl triphenylphosphine bromide (27.81 g of white powder solid) was isolated, yield: 86.44%.

The product does not need to be further purified for following reaction. If pure product is needed, it can be washed with acetone to get white powder solid.

### 4. The Preparation of isovanillin Protected by benzyl Group

Adding isovanillin (15 g, 98.59 mmol ) to anhydrous alcohol (200 ml) in a 250 ml three-necked flask, heating to dissolve at 40° C., adding potassium carbonate (9 g, 65.07 mmol), adding benzylchloride (15 ml, 130.13 mmol) under stirring. The resulting mixture was heated to reflux for 1 hour; After the completion of the reaction (monitored by TLC), cooling it down to 50° C., filtering while hot, cooling the filtrate in refrigeratory overnight, crystal was precipitated, suction filtering, and washing the filter cake with toluene (30 ml). After vacuum drying, the benzyl group protecting isovanillin (white acicular crystal, 19.72 g) was isolated, yield: 82.56%.

The product does not need to be further purified for following reaction. If pure product is needed, it can be recrystallized by absolute alcohol to get white styloid solid.

### 5. The Preparation of cis-trans isomer

Adding 3,4,5-trimethoxyl benzyl triphenylphosphine bromide (20.00 g, 38.21 mmol) and tetrahydrofuran (150 ml) in a 250 ml three-necked flask, stirring the suspension, dissolving isovanillin protected by benzyl group (10.00 g, 41.27 mmol) in tetrahydrofuran (70 ml), and adding it to a dropping funnel (100 ml); adding solid potassium t-butoxide (7.46 g, 66.49 mmol) to the reaction flask, when the reaction system turning to sanguine, stirring for 5 minutes at room temperature, slowly adding the solution of isovanillin protected by benzyl group dropwise, and stirring for 20 minutes at room temperature again; After the completion of the reaction (monitored by TLC), the reaction mixture was transferred into a 500 ml separating funnel, adding deionized water (140 ml), the solution being stratified, extracting with diethyl ether (300 ml) twice, collecting the ether layer, drying with anhydrous magnesium sulfate, filtering, and the filter cake was washed with dry ether (50 ml); concentrating the filtrate in rotatory evaporater to dry to get oily product (25 g); adding absolute alcohol to solidify it, a faint yellow solid (12.50 g) was obtained by suction filtering, yield: 80.48%

### 6. The Recrystallization of cis-trans isomer

Adding cis/trans isomer (12.50 g, 30.75 mmol) and anhydrous alcohol (20 ml) in a 50 ml round bottom flask, heating till some solid is dissolved, stirring at room temperature, after suction filtering, the filter cake was washed with dry ether (10 ml), and drying by Infrared lamp to get pure cis/trans isomer (9.27 g) in faint yellow powder, yield: 74.16%.

### 7. The Preparation of Erianin

Dissolving pure cis/trans isomer (5.14 g, 12.56 mmol) in the mixture of ethyl acetate (100 ml) and absolute alcohol (60 ml) in a 250 ml three-necked flask, the solution being faint yellow, adding 5% Pd—C (0.5 g), stirring while passing hydrogen into the mixture, stirring for 1 hour at room temperature, filtering. After filtering the colorless filtrate was concentrated in rotatory evaporater to dry to obtain a oily product (4.05 g), the crude product of Erianin, yield: 100%.

### 8. The Purification of Erianin

Dissolving the crude product of Erianin (4.05 g, 12.72 mmol) in anhydrous alcohol (20 ml) in a 50 ml round bottom flask, filtering the insoluble substance (if any), and leaving it in stillness for white crystal to be separated at room temperature, then standing overnight. When the solvent is completely volatilized, a great quantity of white crystal is separated. After suction filtering, the filter cake was washed with alcohol to afford the white crystal (3.56 g), yield: 100%.

### Screening of Antineoplastic Effect Using Cell Strains In Vitro

### Example Two

### Experiment on the Cytotoxicity of Erianin

MTT, its chemical formula being 3-(4,5-dimethylthiazd-zyl-2,5-diphenyl tetrazolium bromide), can be used by living cells to form formazan compound through reducing reaction. The product is colored and the cell count can be measured by spectrophotometer.

### Material:

Cell strains: hepG2, Lewis, ECV, QGY, CEM, ECA109, BGC

Erianin, provided by Zhejiang Cell Biomedical Research CO., LTD.

### 1. Cell Strains

Single layer or suspension, the concentration of cell suspension prepared being 10-20×10⁴/ml.

### 2. MTT Solution

Concentration 5 mg/ml, dissolved in physiological saline, stored at 4° C., period of validity being 3 weeks

### 3. Solvent

Comprising of 10% sodium dodecyl sulfate, 5% isobutyl alcohol, and 0.02 mol/L HCl solution

### 4. DMEM Cell Culture Fluid

Prepared according to normal practice (comprising inactivated calf serum and proper amount of antibiotics)

### Method:

Taking a 96-hole plate, adding 90 μl/hole the cell suspension of certain density (equaling cells of 1-2×10⁵/hole), simultaneously (for suspended cells) or after being left at 37° C. for 4-6 hours (for single layer cells) adding 10 μl/hole reagent of different concentrations, each concentration with three multiple holes, and adding culture fluid instead in holes of negative control. Besides, setting up 1-2 blank controls without cells and reagent of 100 μl pure culture fluid/hole as the zero hole for equipment adjustment, vibrating to mix up, putting into incubator of 37° C., 5% CO₂ (48 or 72 hr) and adding MTT solution 20 μl/hole, culturing for another 4hrs, adding solvent 100 μl/hole, putting into incubator of 37° C., and testing the photodensity of each hole with wavelength of A570 (may with DG3022 enzymoimmunoassay device). Comparing the average value of each level of concentration with the negative control value to get the inhibitory percentage of each level of concentration (setting up standard antineoplastic medicine of certain concentration as positive control at the same time). The calculating formula is:

IC%=(C−T)/C×100%

T: OD value of the test group . . . C: OD value of the negative control group Then calculating the IC50 value by regression curve according to the IC% of each test reagent of each level of concentration.

### Effectiveness Evaluation:

Represented by half inhibitory concentration (IC50), where for synthesized medicine IC50<10 μg/ml and for plant extractive IC50<50 μg/ml, the antineoplastic inhibitory effect will be recognized.

### Result:

| No. | Cell type | IC 50 |
|---|---|---|
| 1 | hepG2 Cell | 42.75 |
| 2 | Lewis Cell | 20.83 |
| 3 | ECV Cell | 26.81 |
| 4 | QGY Cell | 52.96 |
| 5 | CEM Cell | 9.38 |
| 6 | ECA109 Cell | 7.58 |
| 7 | BGC Cell | 4.95 |

### Experiment of Tumor Inhibition Rate of Murine Model with Lewis Lung Cancer with the Joint Administration of Erianin and DDP

### Example Three

### The Tumor Inhibitory Effect of Joint Administration of Erianin and DDP on Murine Model with Lewis Lung Cancer

### I. Content of Experiment

### 1. Experiment Sample

(1) Erianin (EN): raw material medicine, provided by Zhejiang Cell Biomedical Research CO., LTD. Batch No.: 060817 Dissolved by DMSO (the end concentration of DMSO being 2 ml/kg) and diluted to the concentration needed with physiological saline.
(2) Cisplatin: Cisplatin for injection (DDP), Qilu Pharmaceutical Co., Ltd. Batch No.: 0503008 Dissolved by physiological saline for preparation
(3) Control product: Cyclophosphamide for injection (CTX), Jiangsu Hengrui Pharmaceutical Inc. Co. Batch No.: 04123021Dissolved by physiological saline for preparation.

### 2. Animals and Tumor Cell Strains

(1) mice bearing Lewis Lung cancer tumor C57BL/6J
(2) 70 C57BL/6J mice, male (ZCBC), weighing 20-24 g.

### 3. Experimental Method

(1) Taking tumor from mouse under aseptic condition, grinding and diluting it by physiological saline 1:3 (weight:volume), and giving hypodermic inoculation 0.2 ml per mouse in the armpit of the right forelimb of each mouse. Dividing the mice into 7 groups randomly, 10 in each group.
(2) After inoculation, waiting till the tumor has grown to the accessible size, administering medicine according to body weight, 0.5 ml/20 g intravenous, for 10 days, putting the mice to death on the 19^{th} day after inoculation, taking out and weighing the tumor, and calculating the tumor inhibiting rate.
(3) Dosage (mg/kg/d) and means of administration:

| | | | Number | | |
|---|---|---|---|---|---|
| Group | | | of | | Administration |
| No. | Medicine | Dosage | animal | Sex | plan |
| Group 1 | EN | 50 mg/kg | 10 | Male | i.p. × 10 qd |
| Group 2 | EN | 100 mg/kg | 10 | Male | i.p. × 10 qd |
| Group 3 | EN | 200 mg/kg | 10 | Male | i.p. × 10 qd |
| Group 4 | DDP | 1 mg/kg | 10 | Male | i.p. × 10 qd |
| Group 5 | EN + DDP | 50 mg/kg + | 10 | Male | i.p. × 10 qd |
| | | 1 mg/kg | | | |
| Group 6 | CTX | 30 mg/kg | 10 | Male | i.p. × 10 qd |
| Group 7 | DMSO | 2 ml/kg | 10 | Male | i.p. × 10 qd |

(4) Judging the result with the following formula:

$$Tumor inhibiting rate = \frac{\begin{matrix}Tumor weight of control group - \\ tumor weight of administrated group\end{matrix} }{Tumor weight of control group} \times 100 \%$$

### II. Experiment Result

The experiment result for inhibiting tumor in mice bearing Lewis lung cancer by joint administration of EN and DDP is as presented in Table 1.

TABLE 1
The experiment result for inhibiting tumor in mice bearing Lewis
lung cancer by joint administration of EN and DDP

| | | | | | | Tumor |
|---|---|---|---|---|---|---|
| Group | | | Means of | Number of | Tumor | inhibiting |
| No. | Sample | Dosage mg/kg | administration | animal | weight | rate |
| Group 1 | EN | 50 | i.p. | 5/5 | 1.59 ± 0.26 | 38.8% |
| Group 2 | EN | 100 | i.p. | 5/5 | 1.40 ± 0.31 | 46.0% |
| Group 3 | EN | 200 | i.p. | 5/5 | 1.15 ± 0.14 | 55.6% |
| Group 4 | DDP | 1 | i.p. | 5/5 | 1.26 ± 0.15 | 51.4% |
| Group 5 | EN + DDP | 12.5 ± 1 | i.p. | 5/5 | 0.99 ± 0.12 | 62.1% |
| Group 6 | CTX | 30 | i.p. | 5/5 | 0.96 ± 0.13 | 63.2% |
| Group 7 | Solvent | 0.04% DMSO | i.p. | 5/5 | 2.6 ± 0.34 | |
| | control | | | | | |

### III. Result Assessment

It is indicated that Erianin is quite effective in inhibiting the tumor in mice bearing Lewis lung cancer, that the joint administration of Erianin and DDP in treating liver cancer showed excellent coordinated curative effect, and that the effect of joint administration is not only more obvious than that of the separate administration of DDP, but also reaches the similar tumor inhibiting effect of the positive control group (CTX).

Wherein, the mice in group 5 had normal appetite, bright hair and free movements. No other abnormality was detected and no death occurred. The mice in group 6 showed the signs of athrepsia, listlessness, and pilo-erection. This phenomenon indicated that the toxicity of the joint administration of Erianin and DDP to mice is lower than that of the sole administration of cyclophosphamide.

All changes based upon the experiments in this invention and all the applicable solid tumors are within the claims of this invention. The above description of the embodiments of this invention does not limit this invention. Those skilled in the art can make various changes and transfigurations according to this invention, and such changes and transfigurations will be within the scope of the claims of this invention as long as they do not get away from the spirit of this invention.

What is claimed is:

1. A use of Erianin represented by formula (I) in preparing a pharmaceutical for treating tumors

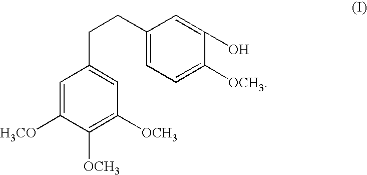

2. The use of claim 1, wherein said tumor is solid tumor.

3. The use of claim 2, wherein said solid tumor is sarcoma and cancer.

4. The use of claim 3, wherein said sarcoma and cancer are selected from a group consisting of fibrosarcoma, myxosarcoma, liposarcoma, chondrosarcoma, osteogenic sarcoma, notochordoma, hemangiosarcoma, endotheliosarcoma, lymphangiosarcoma, lymphangioendotheliosarcoma, synovial bursa, mesothelioma, Ewing's tumor, leiomyosarcoma, rhabdomyosarcoma, carcinoma of colon, carcinoma of pancreas, breast cancer, oophoroma, prostatic carcinoma, squamous carcinoma, basaloma, adenocarcinoma, syringocarcinoma, sebaceous carcinoma, papillary carcinoma, papillary adenocarcinoma, cystadenocarcinoma, encephaloid carcinoma, bronchiolar carcinoma, renal cell carcinoma, liver neoplasm, cholangiocarcinoma, choriocarcinoma, seminoma, embryonal carcinoma, Wilms' tumor, cervical carcinoma, uterine cancer, carcinoma of testis, lung cancer, small cell carcinoma of lung, carcinoma of urinary bladder, epithelial cancer, glioma, astrocytic glioma, acoustic tumor, oligodendroglioma, neurinoma, meningeoma, melanoma, neuroblastoma and retinoblastoma.

5. The use of claim 4, wherein said tumor is sarcoma or lung cancer.

6. A use of Erianin represented by formula (I) in combination with an antitumor agent in preparing pharmaceutical for treating tumors

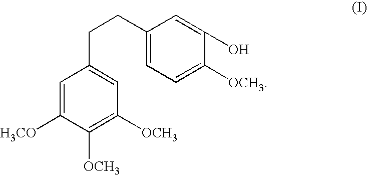

7. The use of claim 6, wherein said antitumor agent is a chemotherapeutic agent.

8. The use of claim 7, wherein said chemotherapeutic agent is Cisplatin, fluorouracil, adriamycin, leuleran, melphalan, taxol, irinotecan, Avastin, N-acetylcochinol-O-phosphate, vascular endothelial-cadherin antibodies anti-VE-cadherin or against VE-cadherin.

9. The use of claim 8, wherein said chemotherapeutic agent is Cisplatin.

10. A method of treatment of tumors comprising administering to a subject in need of such treatment an effective amount of compound represented by formula (I):

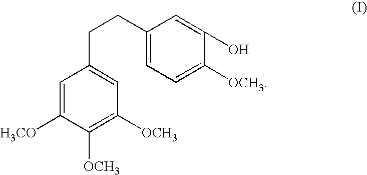

11. A method of treatment of tumors comprising administering to a subject in need of such treatment an effective amount of compound represented by formula (I) in combination with other antitumor agent [chemical structure]
